(19) United States
(12) Patent Application Publication
(10) Pub. No.: US 2020/0368146 A1
(43) Pub. Date: Nov. 26, 2020

(54) METHOD OF COSMETIC PRESERVATION

(71) Applicant: MARY AHERN, Chatham, NY (US)

(72) Inventor: MARY AHERN, Chatham, NY (US)

(21) Appl. No.: 16/821,402

(22) Filed: Mar. 17, 2020

Related U.S. Application Data
(60) Provisional application No. 62/851,477, filed on May 22, 2019.
(60) Provisional application No. 62/879,785, filed on Jul. 29, 2019.

(51) Int. Cl. A61K 8/99; A61K 8/92; A61K 8/64; A61K 8/73; A61Q 19/00

(52) U.S. Cl. CPC A61K 8/99; A61K 8/922; A61Q 19/00; A61K 8/732; A61K 8/645

(57) ABSTRACT

Provided is a method for making a composition for application to skin with improved antimicrobial properties. The method of making a composition for application to skin includes: combining a raw material, water, and a first agent in a first amount effective to inhibit microbial growth to obtain a first mixture; and mixing a second agent in a second amount effective to inhibit microbial growth and the first mixture to obtain a second mixture, wherein a temperature of the second agent is maintained at equal to or less than about 33° C. A composition for application to skin is also provided. The composition for application to skin includes a first agent in a first amount effective to inhibit microbial growth and a second agent in a second amount effective to inhibit microbial growth. The first agent may include a fermentate. The second agent may include a probiotic. The composition for application to skin may further include at least one essential oil and/or at least one terpene.

### RELATED APPLICATION INFORMATION

This patent application is based on and claims priority to U.S. Provisional Application Ser. No. 62/851,477, filed May 22, 2019 and U.S. Provisional Application Ser. No. 62/879,785, filed Jul. 29, 2019, the entire contents of which are herein incorporated by reference.

### TECHNICAL FIELD

The presently disclosed subject matter relates to a method of making a composition for application to skin with improved antimicrobial properties, a composition for application to skin with improved antimicrobial properties and stability, and a method of treating acute skin infection using the composition for application to skin. The composition for application to skin may be a cosmetic composition.

### BACKGROUND

Cosmetic compositions that contain water and organic/inorganic compounds require preservation against microbial contamination to guarantee consumer safety and increase shelf life. Preservation strategies used within the cosmetic industry have proven to be either toxic to the consumer or ineffective in controlling microorganisms. Current regulations limit or prohibit the most potent preservatives due to their toxicity and yet requires uncontaminated cosmetic products. As a result, cosmetic manufacturers are seeking new preservation strategies to present a more secure product in terms of microbiological and toxicological aspects.

The preservation methods of the present disclosure may be utilized in any compositions for application to skin, including cosmetic compositions that contain water.

To the best of the inventor's knowledge, the methods of preservation according to the present disclosure have never been used within the cosmetic industry. As a complete system, it allows compatibility with natural emulsifiers and natural humectants to control water availability in creating emulsions. The methods according to the present disclosure do not require complexity of formulation through inclusion of synthetic or chemical, inherently toxic, ingredients.

Introduction of the preservation methods according to the present disclosure may solve antimicrobial elements of product stability while supporting skin health through improvements in the integrity of epithelial cell tight junctions. In addition, the present consumer need for natural cosmetics has grown, and is expected to grow further, at a rapid rate. The discovery of the present inventor may allow the cosmetic industry to meet the demands of a growing consumer base.

### SUMMARY

An aspect of the present disclosure relates to a method of making a cosmetic composition, comprising: combining a raw material, water, and a first agent in a first amount effective to inhibit microbial growth to obtain a first mixture; and mixing a second agent in a second amount effective to inhibit microbial growth and the first mixture to obtain a second mixture, wherein a temperature of the second agent is maintained at equal to or less than about 33° C. The first agent may comprise a fermentate, a cultured sugar, an antimicrobial protein, an antimicrobial peptide, or combinations thereof. The second agent may comprise a probiotic, including lactic acid bacteria, preferably Bifidobacterium lactis. The composition may further include at least one essential oil and/or at least one relevant terpene.

Another aspect of the present disclosure relates to a composition for application to skin, comprising a first agent in a first amount effective to inhibit microbial growth and a second agent in a second amount effective to inhibit microbial growth. The first agent may include a fermentate. The second agent may include a probiotic. The composition for application to skin may further include at least one essential oil and/or at least one relevant terpene. The relevant terpene may be an isolated terpene.

Another aspect of the present disclosure relates to a method of treating acute skin infection, comprising administering an effective amount of the cosmetic composition according to the present disclosure to a subject in need thereof.

### BRIEF DESCRIPTION OF THE DRAWINGS

FIG. 1 shows the microbial counts for the emulsions of Example 3. The horizontal axis indicate time (days) and the vertical axis indicates Microbial Count (CFU/gm).

FIG. 2 shows the Staph aureus (SA) test results for the samples of Example 4. The horizontal axis indicates time (days) and the vertical axis indicates Microbial Count (CFU/gm).

FIG. 3 shows the Candida parapsilosis (CP) test results for the samples of Example 4. The horizontal axis indicates time (days) and the vertical axis indicates Microbial Count (CFU/gm).

FIG. 4 shows the Staph aureus test results of the serum of Example 6. The horizontal axis indicates time (days) and the vertical axis indicates Microbial Count (CFU/gm).

FIG. 5 shows the Staph aureus test results of the exfoliant of Example 6. The horizontal axis indicates time (days) and the vertical axis indicates Microbial Count (CFU/gm).

FIG. 6 is a photograph of sample products according to the present disclosure taken approximately one month, one year and three weeks, respectively after the sample products were prepared. From left to right: Serum, Exfoliant, Exfoliant+HN019/MG200.

FIG. 7 shows the Staph aureus test results of the emulsions of Example 6. The horizontal axis indicates time (days) and the vertical axis indicates Microbial Count (CFU/gm).

FIG. 8 shows the Staph aureus test results of the evening emulsions of Example 6. The horizontal axis indicates time (days) and the vertical axis indicates Microbial Count (CFU/gm).

FIG. 9 shows the yeast test results of the evening emulsions of Example 6. The horizontal axis indicates time (days) and the vertical axis indicates Microbial Count (CFU/gm).

FIG. 10 shows the Staph aureus test results of the morning emulsions of Example 6. The horizontal axis indicates time (days) and the vertical axis indicates Microbial Count (CFU/gm).

FIG. 11 shows the yeast test results of the morning emulsions of Example 6. The horizontal axis indicates time (days) and the vertical axis indicates Microbial Count (CFU/gm).

FIG. 12 shows a comparison of the Staph aureus test results of the morning emulsion and the evening emulsion of Example 6 and the impact of natamycin. The horizontal axis indicates time (days) and the vertical axis indicates Microbial Count (CFU/gm).

FIG. 13 shows a comparison of the yeast test results of the morning emulsion and the evening emulsion of Example 6 and the impact of essential oils. The horizontal axis indicates time (days) and the vertical axis indicates Microbial Count (CFU/gm).

### DETAILED DESCRIPTION OF THE INVENTION

Illustrative and non-limiting embodiments and examples of the methods and compositions according to the present disclosure are described below in detail. Unless otherwise indicated, the percentage (%) at each occurrence is determined by using weight (as indicated in gm) for solid materials and using volume for liquid materials (as indicated by ml).

An aspect of the present disclosure relates to a method of making a composition for application to skin, comprising: combining a raw material, water, and a first agent in a first amount effective to inhibit microbial growth to obtain a first mixture; and mixing a second agent in a second amount effective to inhibit microbial growth and the first mixture to obtain a second mixture, wherein a temperature of the second agent is maintained at equal to or less than about 33° C.

The first agent may comprise an antifungal agent. The first agent may comprise a fermentate, a cultured sugar, an antimicrobial protein, an antimicrobial peptide, or combinations thereof. The fermentate may be a fermentation product of a composition comprising lactic acid bacteria. The fermentate may be MicroGARD® 200 (Dupont Nutrition & Health, Delaware Del.). Examples of the first agent include cultured sugars, examples of which include but are not limited to lactose, dextrose, fructose and maltodextrin, proteins, examples of which include but are not limited to casein, whey, soy and pea, antimicrobial peptides, and combinations thereof.

The second agent may comprise a probiotic. The probiotic may comprise lactic acid bacteria. Some examples of the lactic acid bacteria include Bifidobacterium lactis, Lactobacillus acidophilus, Lactobacillus plantarum and Lactobacillus paracasei. The probiotic may comprise Bifidobacterium bacteria, Lactobacillus bacteria, Lactococcus bacteria, Streptococcus bacteria, Pediococcus bacteria, and combinations thereof. Preferably, the probiotic comprises Bifidobacterium lactis HN019. The probiotic may also improve the antioxidant system and the ability to decrease radical generation, therefore supporting another aspect of stability within the formula. It may also improve the sensory aspects including smell/odor and improve texture and emulsification of the composition.

The first amount of the first agent effective to inhibit microbial growth is not particularly limited. The first amount of the first agent effective to inhibit microbial growth may be an amount effective to inhibit the growth of mold or yeast. The first amount of the first agent effective to inhibit microbial growth may be about 0.05% or more, about 0.25% or more, about 0.5% or more, about 0.75% or more, about 1% or more, or from about 2.75% or more, and may be about 5% or less, about 4.5% or less, about 4% or less, or about 3% or less by weight (as indicated by gm) relative to the total volume (as indicated by ml) of the composition for application to skin. The first amount of the first agent effective to inhibit microbial growth may be, for example, about 0.25% to about 5%, or from about 0.75% to about 4%, or from about 1% to about 3% by weight relative to the total amount of the composition for application to skin.

A second agent in a second amount effective to inhibit microbial growth may be added into the first mixture to obtain the second mixture, or the first mixture may be added into the second agent in a second amount effective to inhibit microbial growth to obtain the second mixture, or the second agent and the first mixture may be added simultaneously to obtain the second mixture.

The second amount effective to inhibit microbial growth for the second agent is not particularly limited. The second amount effective to inhibit microbial growth may be about 0.10% or more, about 0.25% or more, about 0.37% or more, or about 0.50% or more, and may be about 5% or less, about 4% or less, about 2% or less, or about 1% or less by weight relative to the total amount of the composition for application to skin. The second amount effective to inhibit microbial growth may be, for example, from about 0.10% to about 5%, about 0.20% to about 4%, or about 0.25% to about 1% by weight (as indicated by gm) relative to the total volume (as indicated by ml) of the composition for application to skin.

After the second agent such as a probiotic is added, a temperature of the second agent may be maintained at equal to or less than about 33° C. equal to or less than about 33° C., equal to or less than about 32° C., equal to or less than about 31° C., equal to or less than about 30° C., or equal to or less than about 29° C. For example, the temperature of the second mixture and the temperature at which each of one or more subsequent step is carried out may be maintained at equal to or less than about 33° C. equal to or less than about 33° C., equal to or less than about 32° C., equal to or less than about 31° C., equal to or less than about 30° C., or equal to or less than about 29° C. For example, the method of making a composition for application to skin may comprise emulsification of a composition containing the probiotic and the temperature at the end of the emulsification of the composition containing the probiotic may be controlled to be equal to or less than about 33° C., equal to or less than about 32° C., equal to or less than about 31° C., equal to or less than about 30° C., or equal to or less than about 29° C.

The method of making a composition for application to skin may further comprise mixing at least one essential oil and/or at least one relevant terpene and the second mixture. The relevant terpene may be an isolated terpene. For example, the at least one essential oil and/or the at least one terpene may be added into the second mixture. Alternatively, the second mixture may be added into the at least one essential oil and/or the at least one terpene. Alternatively, the second mixture and the at least one essential oil and/or the at least one terpene may be added simultaneously, for example, into a container. Examples of the at least one essential oil include an essential oil from the Burceraceae family, including Frankincense (Boswellia sacra, B. carterii, B. frereana, B. serrata and B. papyrifera), Palo santo (Bursera graveolens), Myrrh (Commiphora Myrrha or Commiphora Molmol) and Copal (Protium copal), essential oils Tulsi or Holy basil (Ocimum tenuiflorum), Turmeric (Curcuma longa), Vetiver (Vetiveria zizanioides), Hemp (Cannabis salvia) including seed oil, and essential oils derived from the genus Citrus (Rutaceae) (examples include Citrus Sinensis, Citrus uranium, Citrus reticulata, Citrus lemon, Citrus aurantifolia, Citrus×paradise, Citrus bergamia, Citrus junos, Citrus japonica) and combinations thereof. For example, a combination of the following essential oils may be used: Vetiver, Palo santo, Holy basil, Frankincense and Lime. In certain embodiments, that at least one essential oil contains a terpene, for example, limonene, alpha pinene, caryophyllene, myrcene, sabinene, eugenol, beta-Elemene, alpha-Bulsene, gamma terpinene, terpinolene and menthofuran. That at least one essential oil, or a combination of one or more essential oils, and/or inclusion of related terpenes, may play a symbiotic role in the composition for application to skin.

The amount of the at least one essential oil and/or the at least one terpene may be about 0.2% or more, about 0.5% or more, about 1% or more, about 2% or more, or about 2.6% or more, and may be about 10% or less, about 5% or less, about 4% or less, or about 3% or less by volume relative to the total volume of the composition for application to skin. The amount of at least one essential oil and/or the at least one terpene may be, for example, from about 0.2% to about 10%, about 0.5% to about 4%, or about 1% to about 3% by volume relative to the total volume of the composition for application to skin. For example, when the composition for application to skin is an emulsion, the amount of the at least one essential oil and/or the at least one terpene may be about 2.57% by volume relative to the total volume of the composition. When the composition for application to skin is a serum, the amount of the at least one essential oil and/or the at least one terpene may be about 2.6% by volume relative to the total volume of the composition. When the composition for application to skin is an exfoliant, the amount of the at least one essential oil and/or the at least one terpene may be about 1.14% by volume relative to the total volume of the composition. When more than one essential oils and/or terpenes are used, the amount of the at least one essential oil and/or the at least one terpene described in this disclosure means the total amount of the more than one essential oils and/or terpenes.

In an embodiment, the method of making a composition for application to skin according to the present disclosure may comprise:
(1) combining a fermentate and a water element to obtain a first composition;
(2) combining an oil element and a wax element, and heating to about 63° C. to about 74° C. to obtain a second composition;
(3) adding a glycerite extraction to the first composition and agitating to obtain a third composition;
(4) adding a probiotic to the third composition to obtain the fourth composition;
(5) adding the second composition that has been cooled to about 60° C., to the fourth composition and emulsifying at a temperature of less than or equal to about 33° C.;
(6) cooling the product of step (5) to less than or equal to 18° C.; and
(7) adding essential oils to the product of step (6).

The composition for application to skin may be a cream. An example of the method of making the composition for application to skin may comprise:
(1) combining a fermentate (about 0.75% to about 4%) with a water element (about 50%) and agitating;
(2) combine an oil element (about 40%) with a wax element (about 3%) and heating to about 63-74° C.;
(3) adding a glycerite extraction (about 2%) to the product of step (1) and agitating;
(4) adding a probiotic (about 0.25% to about 1%) to the product of step (3).
(5) adding the oil/wax composition obtained in step (2) that has been cooled to 60° C., to the water/fermentate/glycerite/probiotic composition obtained in step (4), and agitating/blending to emulsify at less than or equal to about 33° C.;
(6) cooling the mixture to less than or equal to 18° C. and more than 0° C.; and
(7) adding essential oils (about 2%) to the product of step (6) and agitating until incorporated.
   The final product may be stored away from light at about 10° C. to about 18° C.

In another embodiment, the method of making a composition for application to skin according to the present disclosure may comprise:
(1) agitating a fermentate into a liquid composition comprising water and glycerine to obtain a first composition;
(2) agitating a probiotic into the first composition to obtain the second composition; and
(3) adding at least one essential oil to the second composition.

In the embodiment, the method may be carried out at a temperature of less than or equal to 21° C.

The composition for application to skin may be a liquid composition, such as a serum. An example of the method of making the liquid composition may be carried out at a temperature of less than or equal to 21° C. and may comprise:
(1) combining liquid ingredients, such as glycerite extractions (water about 28%, glycerine about 66%);
(2) agitating a fermentate (about 0.75% to about 4%) into the composition obtained in step (1);
(3) agitating a probiotic (about 0.25% to about 1%) into the composition obtained in step (2); and
(4) adding at least one essential oil (about 2.50%).
   The obtained final product may be stored away from light at about 10° C. to about 18° C.

In another embodiment, the method of making a composition for application to skin according to the present disclosure may comprise:
(1) combining dry ingredients, the probiotics and the fermentate to obtain a first composition;
(2) adding the first composition to a liquid component comprising water and glycerine and agitating to obtain a second composition;
(3) combining a dry plant ingredient or a clay ingredient, and the second composition, and agitating to obtain a third composition;
(4) adding a humectant to the third composition and agitating to obtain a fourth composition; and
(5) adding essential oils to the fourth composition and mixing to an incorporated consistency.

Another example of the method of making the liquid composition may be carried out at a temperature of less than or equal to 21° C. and may comprise:
(1) combining liquid ingredients, for example, a glycerite extraction (water about 11%, glycerine about 26%);
(2) combining dry milk based ingredients (about 1.35%) with a probiotics (about 0.25% to about 1%) and a fermentate (about 0.75 to about 4%), adding to the composition obtained in step (1), and agitating;
(3) combining dry plant or clay ingredients (about 14%) and adding to the composition obtained in step (2), and agitating;
(4) adding a humectant, specifically, but not limited to, honey (about 45%) to the composition obtained in step (3) and agitating; and
(5) add at least one essential oil (about 1.25%) and agitating to mix to an incorporated consistency.
   The obtained final product may be stored away from light at about 10° C. to about 20° C.

Another aspect of the present disclosure relates to a composition for application to skin, comprising a first agent in a first amount effective to inhibit microbial growth and a second agent in a second amount effective to inhibit microbial growth.

The first agent may comprise a fermentate, a cultured sugar, an antimicrobial protein, an antimicrobial peptide, or combinations thereof. The fermentate may be a fermentation product of a composition comprising lactic acid bacteria. The fermentate may be MicroGARD® 200 (Dupont Nutrition & Health, Delaware, Del.). Examples of the first agent include cultured sugars, examples of which include but are not limited to lactose, dextrose, fructose and maltodextrin, proteins, examples of which include but are not limited to casein, whey, soy and pea, antimicrobial peptides, and combinations thereof.

The second agent may comprise a probiotic. The probiotic may comprise lactic acid bacteria. Examples of the lactic acid bacteria include Bifidobacterium lactis, Lactobacillus acidophilus, Lactobacillus plantarum and Lactobacillus paracasei. The probiotic may comprise Bifidobacterium bacteria, Lactobacillus bacteria, Lactococcus bacteria, Streptococcus bacteria, Pediococcus bacteria, and combinations thereof. Preferably, the probiotic comprises Bifidobacterium lactis HN019. The probiotic may also improve the antioxidant system and the ability to decrease radical generation, therefore supporting another aspect of stability within the formula. It may also improve sensory aspects including smell/odour and improve texture and emulsification of the composition.

The first amount effective to inhibit microbial growth for the first agent is not particularly limited. The first amount effective to inhibit microbial growth for the first agent is not particularly limited. The first amount effective to inhibit microbial growth may be an amount effective to inhibit the growth of mold or yeast. The first amount of the first agent effective to inhibit microbial growth may be about 0.05% or more, about 0.25% or more, about 0.5% or more, about 0.75% or more, about 1% or more, or from 2.75% or more, and may be about 5% or less, about 4.5% or less, about 4% or less, or about 3% or less by weight (as indicated by gm) relative to the total volume (as indicated by ml) of the composition for application to skin. The first amount of the first agent effective to inhibit microbial growth may be, for example, about 0.25% to about 5%, or from about 0.75% to about 4%, or from about 1% to about 3% by weight relative to the total amount of the composition for application to skin.

The second amount effective to inhibit microbial growth for the second agent is not particularly limited. The second amount effective to inhibit microbial growth may be about 0.10% or more, about 0.25% or more, about 0.37% or more, or about 0.50% or more, and may be about 5% or less, about 4% or less, about 2% or less, or about 1% or less by weight relative to the total amount of the composition for application to skin. The second amount effective to inhibit microbial growth may be, for example, from about 0.10% to about 5%, about 0.20% to about 4%, or about 0.25% to about 1% by weight (as indicated by gm) relative to the total volume (as indicated by ml) of the composition for application to skin.

Preferably, the first agent is a fermentate and the first amount effective to inhibit microbial growth is from about 0.75% to about 4%, or about 1% to about 3% by weight relative to the total amount of the composition for application to skin.

Preferably, the second agent is a probiotic and the second amount effective to inhibit microbial growth is from about 0.25% to about 1% by weight relative to the total amount of the composition for application to skin.

The composition for application to skin may further comprise at least one essential oil and/or at least one terpene relevant to preventing microbial growth. The at least one terpene may be at least one isolated terpene. Examples of the at least one essential oil include an essential oil from the Burceraceae family, including Frankincense (Boswellia sacra, B. carterii, B. frereana, B. serrata and B. papyrifera), Palo santo (Bursera graveolens), Myrrh (Commiphora Myrrha or Commiphora Molmol) and Copal (Protium copal), essential oils Tulsi or Holy basil (Ocimum tenuiflorum), Turmeric (Curcuma longa), Vetiver (Vetiveria zizanioides), Hemp (Cannabis salvia) including seed oil, and essential oils derived from the genus Citrus (Rutaceae) (examples include Citrus Sinensis, Citrus uranium, Citrus reticulata, Citrus lemon, Citrus aurantifolia, Citrus×paradise, Citrus bergamia, Citrus junos, Citrus japonica) and combinations thereof. For example, a combination of the following essential oils may be used: Vetiver, Palo santo, Holy basil, Frankincense and Lime. In certain embodiments, that at least one essential oil contains a terpene, for example, limonene, alpha pinene, caryophyllene, myrcene, sabinene, eugenol, beta-Elemene, alpha-Bulsene, gamma terpinene, terpinolene and menthofuran. That at least one essential oil, or a combination of one or more essential oils, and/or inclusion of related terpenes, may play a symbiotic role in the composition for application to skin.

The amount of the at least one essential oil and/or the at least one terpene may be about 0.2% or more, about 0.5% or more, about 1% or more, about 2% or more, or about 2.6% or more, and may be about 10% or less, about 5% or less, about 4% or less, or about 3% or less by volume relative to the total volume of the composition for application to skin. The amount of the at least one essential oil and/or the at least one terpene may be, for example, from about 0.2% to about 10%, about 0.5% to about 4%, or about 1% to about 3% by volume relative to the total volume of the composition for application to skin. For example, when the composition for application to skin is an emulsion, the amount of the at least one essential oil and/or the at least one terpene may be about 2.57% by volume relative to the total volume of the composition. When the composition for application to skin is a serum, the amount of the at least one essential oil and/or the at least one terpene may be about 2.6% by volume relative to the total volume of the composition. When the composition for application to skin is an exfoliant, the amount of the at least one essential oil and/or the at least one terpene may be about 1.14% by volume relative to the total volume of the composition. When more than one essential oils and/or terpenes are used, the amount of the at least one essential oil and/or the at least one terpene described in this disclosure means the total amount of the more than one essential oils and/or terpenes.

Another aspect of the present disclosure relates to a method of treating acute skin infection, comprising administering an effective amount of the composition for application to skin according to the present disclosure to a subject in need thereof. The effective amount of the composition may be an amount that is sufficient to ameliorate, alleviate or improve a symptom of the acute skin infection. The method of treating acute skin infection may involve administration of the composition over a prolonged period of time. For example, the composition for application to skin according to the present disclosure may be administered to the subject over a period of at least 8 hours, over a period of at least 12 hours, over a period of at least one day, over a period of at least one week, or over a period of at least two weeks.

### EXAMPLES

### Example 1

Preservation trials began with the utilization of salt to control undesirable microbial activity by creating an environment not conducive to microbial growth. For this trial, three treatments were evaluated: a control with no salt, low salt (0.05%) and high salt (5%). Additionally, HOLDBAC® LC (Dupont Nutrition & Health, Delaware, Del.) was added at 0.093% to determine if this antimicrobial along with the salt would give desired results. The initial results are shown in Table 1. The one-month results are shown in Table 2.

TABLE 1
Salt Trial: Initial results

| Percent | HOLDBAC ® | Water | TPC | | Y/M | |
|---|---|---|---|---|---|---|
| Salt | LC | Activity | (Total Plate Count) | | (Yeast/Mold) | Comments |
| 0.0% | 0.093% | 0.886 | 50 | CFU/gm | Clean | |
| 0.5% | 0.092% | 0.883 | 5.6 × 10e5 | CFU/gm | 20 cfu/gm Yeast | |
| | | | | | 440 cfu/gm Mold | |
| 5.0% | 0.088% | 0.884 | 4.1 × 10e2 | CFU/gm | 200 cfu/gm Yeast | Quality |
| | | | | | 960 cfu/gm Mold | affected |
| | | | | | | detrimentally |

Salt used - Organic Sea Salt to support clean labeling.

These results confirm clean production. It appears that bacteria, yeast and mold were introduced with the addition of salt.

TABLE 2
Salt Trial: one-month results

| | Water | | | | |
|---|---|---|---|---|---|
| Percent | Activity | Yeast | Mold | Yeast | Mold |
| Salt | (Aw) | T = 0 | T = 0 | T = 1 | T = 1 |
| 0 | 0.886 | <10 | <10 | <10 | <10 |
| 0.50% | 0.883 | 2.00E+01 | 4.40E+02 | 2.80E+02 | 3.00E+01 |
| 5.00% | 0.884 | 2.00E+02 | 9.60E+02 | 2.20E+02 | <10 |

### Results and Discussion.

Salt was commonly used in natural preservation and suggested to be a good and obvious starting point. Our results in this experiment showed that we were able to achieve clean production, and that salt did not play a role in controlling Yeast, Mold or TPC. In fact, salt appears to have played a detrimental role by introducing undesirable bacteria and Y/M at time of manufacture. In conclusion, salt is not working to control microbial contamination and we have chosen to no longer investigate this treatment as a possible solution.

It was also decided that it was not possible to determine if the HOLDBAC® LC was playing a role in these trials due to the confounding factors introduced by salt. More trials with HOLDBAC® LC are needed to determine its efficacy.

It was decided that legitimate challenge testing would be necessary to determine if the formula would support the growth of these potential contaminants.

### Example 2: Challenge Study #1

Two samples of an emulsion formula were produced as follows:
(1) provide the water element (about 50%),
(2) Combine oil element (about 40%) with wax element (about 3%), heat to about 63-74 degrees Celsius;
(3) Add glycerite extractions, (about 2%) to water element (1), agitate;
(4) Add probiotics (Holdbac LC which is a probiotic, Lactobaccillus rhamnosis, was added with HN019. LC was added at 0.09% and HN019 at 0.17%) to the mixture obtained in (3);
(5) Add oil/wax obtained in (2), cooled to 60 degrees Celsius, to the mixture obtained in (4), and agitate to emulsify less than or equal to 33 degrees Celsius;
(6) Add essential oils (about 2%), and agitate prior to cooling; and
(7) Cool mixture to less than or about 18 degrees Celcius (and do not freeze).

The samples were stored away from light at about 10 to 18 degrees Celsius.

The two cosmetic cream samples, 1110001-MML Emulsion N92117 and 1110002-MML Emulsion E100517, were sent to Biogen Laboratory Developments, LLC for challenge testing using Staph. Aureus and Candida parapsilosis. It was suggested by this laboratory that these two microorganisms would provide sufficient bioburden to assess the MML formulations. Trials began with emulsions as these products are most susceptible to microbial contamination issues. In challenge study #1, two emulsion samples were tested. The initial microbial load of each of these uninoculated products was found to be free of bacteria, yeast and mold, as seen in Table 3 below.

TABLE 3
Challenge Study #1. Plate count results for uninoculated product

| | Aerobic | Yeast & Mold |
|---|---|---|
| Results: | Plate Count: | Count: |
| 1110001-MML | <10 CFU/g | <10/<10 CFU/g |
| 1110002-MML | <10 CFU/g | <10/<10 CFU/g |

*CFU = Colony Forming Unit

Each product was inoculated with 24 hr cultures of Staph. aureus (SA) or Candida parapsilosis (CP) respectively. The 24 hr culture was tested to establish the overall bioburden that would be delivered. Below are the results:

Staph. aureus=2.6×10⁸ cfu/ml

Candida parapsilosis=1.5×10⁶ cfu/ml

For each sample, two sub-samples of 50 gm each were inoculated with either Staph. aureus (SA) or Candida parapsilosis (CP) by adding 1.0 ml of a 24-hr culture to the 50 g sub-sample.

Each sub sample inoculated with SA had a final bioburden of 2.6×10⁶ cfu/g of sample.

Each sub sample inoculated with CP had a final bioburden of 1.5×10⁴ cfu/g of sample.

TABLE 4
Challenge Study 1: Microbial counts for MML emulsions

| Sample | Treatment | Bioburden | T = 0 | T = 1 day | T = 2 day |
|---|---|---|---|---|---|
| 1110001 MML | Emulsion - N92117 | SA | 420,000 | 180,000 | 38,000 |
| 1110002 MML | Emulsion - E100517 | SA | 370,000 | 77,000 | 21,000 |
| 1110001 MML | Emulsion - N92117 | CP | 7,100 | 24,000 | 120,000 |
| 110002 MML | Emulsion - E100517 | CP | 9,600 | 25,000 | 48,000 |

### Results and Discussion.

The emulsion N92117 contained Bifidobacterium lactis HN019 (a lactic acid bacteria, commonly known as a probiotic) at 0.17% of the formulation, in addition to HOLDBAC® LC (a probiotic shown to be effective against yeast and mold in the food industry) at 0.09%. Temperature at emulsification was 99° F. (37° C.).

The results are shown in Table 4. Staph. aureus counts dropped in this formulation by more than half within 24 hr and drop by over 1 log on 2-day. This combination had no impact on reducing yeast counts, in fact the counts are increasing over 2.5 logs during the course of this 2-day study. This shows that HOLDBAC® LC is not effective at controlling yeast in these cosmetic formulations.

The emulsion E100517 contained Bifidobacterium lactis HN019 at 0.14% of the formulation, in addition to HOLDBAC® LC at 0.09%. Temperature at emulsification was also 99° F. (37° C.). This emulsion also contained a high level (0.59%) of an orange essential oil. This combination had no impact on yeast. However, there was improvement in count reduction in Staph. aureus, even more significant than N92117 (Table 4). This again confirms that HOLDBAC® LC was not effective at controlling yeast in these cosmetic formulations. It was noted that the addition of the essential oil may be improving the performance in controlling Staph. aureus and may be contributing to the control of yeast. More research needs to be done to understand its role.

The data illustrates a gradual reduction of Staph. aureus ˜1 log within the first 2 days of ambient storage with both formulations. The Candida however increased in counts nearly 1 log within the first 24 hours and nearly another log within 48 hours in the Emulsion N92117. The Emulsion E100517 remained steady at day 2. The overall recovery of these organisms was lower in the E100517 Emulsion indicating it has greater resilience than the N92117 formula. The levels will continue to be monitored to conclude the growth trends of these organisms in these samples.

The data suggests that the Staph aureus may ultimately starve out in both formulas, but that the Candida may continue to grow in both formulations.

### Example 3: Challenge Study #2

This study was designed to test the impact of BioVida (multi-hurtle antimicrobial including mustard seed, green tea). This antimicrobial was selected for its clean label status.

The emulsion sample was prepared as follows:
(1) Combine fermentate (Bio Vida 0.89%) with water element (about 50%),
(2) Combine oil element (about 40%) with wax element (about 3%), heat to about 63-74 degrees Celsius;
(3) Add glycerite extractions, (about 2%) to the water element (1), agitate;
(4) Add probiotics (0.42% HN019) to the mixture obtained in (3);
(5) Add oil/wax obtained in (2), cooled to 60 degrees Celsius, to the mixture obtained in (4), and agitate to emulsify less than or equal to 33 degrees Celsius;
(6) Add essential oils (about 2%), and agitate prior to cooling; and
(7) Cool mixture to less than or about 18 degrees Celcius (and do not freeze).

The samples were stored away from light at about 10 to 18 degrees Celsius.

This study was performed on the cosmetic cream sample 0426001-MML ONPA 41018.

The cream was tested initially to check for background organisms (Table 5).

TABLE 5
Challenge Study 2. Counts for background organisms

| | Rapid Aerobic | Rapid Yeast & |
|---|---|---|
| Results: | Plate Count: | Mold Count: |
| 0426001-MML | <10 CFU/g | <10/<10 CFU/g |

*CFU = Colony Forming Unit

For this sample, two sub-samples of 50 g each were inoculated with either Staph. aureus (SA) or Candida parapsilosis (CP) by adding 0.5 ml of a 24-hour culture of Staph. aureus (SA) or Candida parapsilosis (CP), respectively, to the 50 g sub-sample.

The 24-hour culture was tested to establish how much bacteria was added to the samples.

Staph. aureus: 1.1×10⁸ CFU/ml

Candida parapsilosis: 1.4×10⁷ CFU/ml

Each 50 g sub-sample inoculated with SA received roughly 1,100,000 CFU/g of sample. Each 50 g sub-sample inoculated with CP received roughly 140,000 CFU/g of sample.

Results: The results are shown in FIG. 1 and Table 6 below.

TABLE 6
Microbial counts in Challenge Study 2

| | 0426001 SA | 0426001 CP |
|---|---|---|
| Day 0: | 240,000 | 20,000 |
| Day 1: | 710,000 | 37,000 |
| Day 2: | 250,000 | 37,000 |
| Day 3: | 150,000 | 41,000 |
| Day 4: | 100,000 | 80,000 |
| Day 5: | 40,000 | 120,000 |
| Day 6: | 12,000 | 96,000 |
| Day 7: | 100 | 180,000 |
| Day 14: | 10 | 310,000 |
| Day 21: | 3,700 | 5,500,000 |

In this study we did not see the reduction in Staph. aureus as we have seen in previous experiments. Emulsion temperature was 95 F (35 C). The Bifidobacterium lactis HN019 was added very early in the process. No terpene (Limonene—orange essential oil contains 90%) was used in the formulation. Possible causes could be addition time of Bifidobacterium lactis HN019 and sequence of addition may have an impact as it was introduced earlier than in previous trials that showed better results. Additionally, the lack of terpenes may have played a role. BioVida does not appear to have a positive impact on either the Staph. aureus or Candida parapsilosis.

### Example 4: Challenge Study #3

This experiment compared fermentates (MG100 (dairy organic), MG200 (organic), MG210 (not organic)) and antimicrobial (Natamycin at various levels) and their impact on Staph. aureus and Candida parapsilosis. As process may playing a role in the results, specific processing conditions (sequence and temperature) and assessment of their potential impacts on microbial results will be included in all future studies.

### Study:

MML-1: MEN 70918 (7/9 AM)—MG100

MML-2: EEN 71018 (7/10 AM)—MG200

MML-3: EVEN 71118 (7/11 AM)—MG210

MML-4: Morning Emulsion 81418 (8/14/18 AM)—Natamycin (0.025%)

MML-5: Eve Emulsion 81818 (8/14/18 AM)—Natamycin (0.0125%)

Five creams were prepared by a process similar to Example 3, except for those components, amounts and/or conditions specifically noted in this example. All treatments include Bifidobacterium lactis HN019 at 0.41-0.47%.

Each cream was tested initially to determine background organisms. The products were tested for APC, yeast and mold (Table 7).

TABLE 7
Counts for background organisms in Example 4

| | Rapid Aerobic | Rapid Yeast & |
|---|---|---|
| Results: | Plate Count: | Mold Count: |
| MML-1 | <10 CFU/g | <10/<10 CFU/g |
| MML-2 | <10 CFU/g | <10/<10 CFU/g |
| MML-3 | <10 CFU/g | <10/<10 CFU/g |
| MML-4 | <10 CFU/g | <10/<10 CFU/g |
| MML-5 | <10 CFU/g | <10/<10 CFU/g |

*CFU = Colony Forming Unit

Each product was inoculated with 24 hr cultures of Staph. aureus (SA) or Candida parapsilosis (CP) respectively. The 24 hr culture was tested to establish the overall bioburden that would be delivered.
Staph. aureus=2.8×10⁸ cfu/ml
Candida parapsilosis=3.7×10⁶ cfu/ml

For each sample, two sub-samples of 100 gm each were inoculated with either Staph. aureus (SA) or Candida parapsilosis (CP) by adding 1.0 ml of a 24-hr culture to the 100 g sub-sample.

Each 100 gm sub sample inoculated with SA had a final bioburden of 2.8×10⁶ cfu/g of sample.

Each 100 gm sub sample inoculated with CP had a final bioburden of 37,000 cfu/g of sample.

### Results and Discussion.

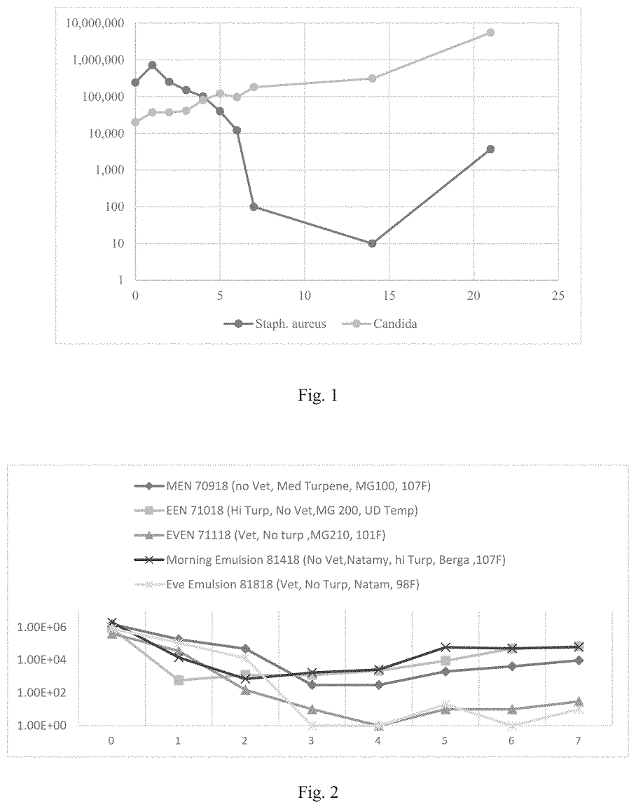

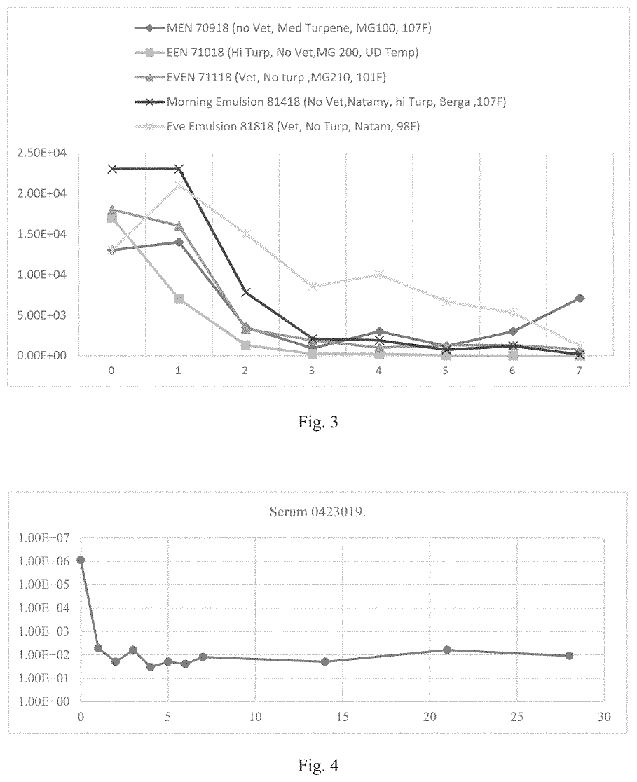

The results of Example 4 are shown in Tables 10-12, as well as in FIG. 2 and FIG. 3.

TABLE 8
Counts for MML-1

| | MML-1 SA | MML-1 CP |
|---|---|---|
| Day 0: | 1,500,000 | 13,000 |
| Day 1: | 180,000 | 14,000 |
| Day 2: | 48,000 | 3,500 |
| Day 3: | 300 | 920 |
| Day 4: | 300 | 3,000 |
| Day 5: | 2,000 | 1,200 |
| Day 6: | 4,100 | 3,000 |
| Day 7: | 9,500 | 7,100 |

TABLE 9
Counts for MML-2

| | MML-2 SA | MML-2 CP |
|---|---|---|
| Day 0: | 800,000 | 17,000 |
| Day 1: | 580 | 7,000 |
| Day 2: | 1,200 | 1,300 |
| Day 3: | 1,200 | 220 |
| Day 4: | 2,200 | 210 |
| Day 5: | 8,900 | 40 |
| Day 6: | 50,000 | 10 |
| Day 7: | 67,000 | <10 |

TABLE 10
Counts for MML-3

| | MML-3 SA | MML-3 CP |
|---|---|---|
| Day 0: | 400,000 | 18,000 |
| Day 1: | 35,000 | 16,000 |
| Day 2: | 150 | 3,300 |
| Day 3: | 10 | 1,900 |
| Day 4: | <10 | 1,000 |
| Day 5: | 10 | 1,300 |
| Day 6: | 10 | 1,300 |
| Day 7: | 30 | 790 |

TABLE 11
Counts for MML-4

| | MML-4 SA | MML-4 CP |
|---|---|---|
| Day 0: | 1,900,000 | 23,000 |
| Day 1: | 14,000 | 23,000 |
| Day 2: | 700 | 7,800 |
| Day 3: | 1,700 | 2,100 |
| Day 4: | 2,600 | 1,900 |
| Day 5: | 59,000 | 740 |
| Day 6: | 50,000 | 1,200 |
| Day 7: | 61,000 | 150 |

TABLE 12
Counts for MML-5

| | MML-5 SA | MML-5 CP |
|---|---|---|
| Day 0: | 800,000 | 13,000 |
| Day 1: | 110,000 | 21,000 |
| Day 2: | 13,000 | 15,000 |
| Day 3: | <10 | 8,500 |
| Day 4: | <10 | 10,000 |
| Day 5: | 20 | 6,700 |
| Day 6: | <10 | 5,300 |
| Day 7: | 10 | 1,200 |

MEN 70918 MG100—Sequence of Bifidobacterium lactis HN019 was correct but processing end temperature (after emulsification) was 107° F. (41.6° C.). This treatment showed increasing Staph. aureus (SA) and Candida parapsilosis (CP) beginning at 4 and 6 weeks respectively. Temperature likely contributed to the failure. Due to the lack of impact by MG100, this ingredient would be removed from further testing.

EEN 71018—The product had terpenes (Orange essential and Ylang Ylang sourced). Though not documented, it is hypothesized that the end temperature on this treatment was also high. The Staph aureus was high, and this may be consistent with other trial when temperature was elevated during emulsification. This treatment had a significant effect of yeast reduction and further studies are warranted to evaluate MG200.

It appears that end temperature after emulsification is relevant on impacting Staph aureus (SA) results. B. lactis HN109 was significantly more effective with temps less than 101° F. (38.3° C.

The essential oil, Vetiveria zizanioides, is showing potential impact as seen in EVEN 71118. With 71118, emulsification temperature was 101 F (38.3 C), sequence of addition was correct and MG210.

Due to the good performance and clean label (certified organic) with MG200, this antimicrobial will be used instead of MG 210 in future studies.

There may be a relationship between MG200 and essential oils. In particular, Vetiveria zizanioides, may be providing a support or symbiotic relationship.

Although Natamycin showed good control of Candida parapsilosis (CP), it had a negative impact on product quality including negative odor and consistency impacts. Natamycin is also not considered a clean label. For these reasons, this ingredient would be eliminated in future experiments.

### Example 5: Challenge Study #4

In this study, three treatments were designed to separate impact plant (hemp) and essential oils. B. lactis HN019 were added to all treatments to support the control of Staph aureus. This was the first studies to evaluate addition of the terpenes after cooling 65° F. (18.3° C.). The concern was that higher temperature could induce cytotoxic elements (they are temperature sensitive).

The samples were prepared by a process similar to Example 3, except for those components, amounts and/or conditions specifically noted in this example.

Treatments:

Morning E Blend 112918 (Vetiveria zizanioides, Essential Oil (Terpenes), Hemp, MG200, 88 F)

Evening Blend 121018 (Vetiveria zizanioides, Essential Oil (Terpenes) added after cooling, Hemp, MG200, 2.5% NCFM, 88 F)

Evening E 112618 (Vetiveria zizanioides, Terpenes added after cooling, Hemp, MG200, 109 F)

112918—Inhibition of Staph aureus by day 4, and yeast to 150 by day 5.

121018—Inhibition of Staph aureus by day 4, up, then <10 by day 7. Yeast counts to 20 by day 7.

112618—Staph aureus poorly controlled at 1,500,000 cfu by day 7, yeast to 10 cfu/g by day 7. Temperature appears to have had a negative impact on the B. lactis HN019. This continues to confirm that temperature will be key to the solution on Staph aureus results.

Conclusion:

Yeast and Staph aureus were controlled in 2 of the 3 treatments. Temperature after emulsification continues to be a relevant factor. For the yeast results, MG200 and the addition of essential oils after cooling appear to be contributing to positive results.

### Example 6: Challenge Study #5

In this study, the role of essential oils in the emulsion formulations using a standardized protocol (sequence, temperature, MG200, and Bifidobacterium lactis HN019) was evaluated. This study also evaluated the role of natamycin at low levels to determine its impact on yeast counts.

The emulsion formulations were prepared by a process similar to Example 3, except for those components, amounts and/or conditions specifically noted in this example. The fermentate was added first to water.

There additional formulas, a serum (containing water) and two exfoliants (containing ingredients that can contribute to microbial contamination) were added to trials to determine if the method of preservation currently showing positive results in the emulsion formulas could be transferred to other cosmetic formulations. Both formulations were produced via a cold process.

The serum was prepared by cool temperature production at less than or equal to 21 degrees Celsius, as follows:
(1) Combine liquid ingredients, specifically glycerite extractions, (water about 28%, glycerine about 66%);
(2) Agitate fermentate MG 200 (about 0.09%) into the liquid obtained in (1);
(3) Agitate 0.49% probiotic B. lactis HN019 and 1.68% Lactobacillus acidophilus NCFM (For Skin Health) into the liquid obtained in (2); and
(4) Add essential oils (about 2.50%).
   The serum was stored away from light at about 10 to 18 degrees Celsius.

The exfoliant was prepared by the process below carried out at a temperature of less than or equal to 21° C.:
(1) combining liquid ingredients, for example, a glycerite extraction (water about 11%, glycerine about 26%);
(2) combining dry milk based ingredients (about 1.35%) with a probiotics (about 0.25% to about 1%) and a fermentate (about 0.75 to about 4%), adding to the composition obtained in step (1), and agitating;
(3) combining dry plant or clay ingredients (about 14%) and adding to the composition obtained in step (2), and agitating;
(4) adding a humectant, specifically, but not limited to, honey (about 45%) to the composition obtained in step (3) and agitating; and
(5) add at least one essential oil (about 1.25%) and agitating to mix to an incorporated consistency.
   The obtained final product was stored away from light at about 10° C. to about 20° C.

Four cream blends, two exfoliants and one serum were provided to challenge them against Staphylococcus aureus and Candida parapsilosis.

Each cream was tested initially to check for background microbial levels (Table 13):

TABLE 13
Background microbial levels in Example 7

| | | Rapid Aerobic | Rapid Yeast & |
|---|---|---|---|
| Sample No. | Results: | Plate Count: | Mold Count: |
| 0510001- | Botanical Serum | 100 CFU/g | <10/<10 CFU/g |
| | 0423019 | | |
| 0510002- | Botanical Exfoliant | 100 CFU/g | <10/<10 CFU/g |
| | 030118 | | |
| 0510003- | Botanical Exfoliant | 440 CFU/g | <10/<10 CFU/g |
| | 050719 | | |
| 0510004- | Botanical Emulsion | <10 CFU/g | <10/<10 CFU/g |
| | Evening | | |
| 0510005- | Botanical Emulsion | <10 CFU/g | <10/<10 CFU/g |
| | Evening | | |
| 0510006- | Botanical Emulsion | <10 CFU/g | <10/<10 CFU/g |
| | Morning | | |
| 0510007- | Botanical Emulsion | <10 CFU/g | <10/<10 CFU/g |
| | Morning | | |

*CFU = Colony Forming Unit

For each sample, two sub-samples of each were inoculated at 1% with either Staphylococcus aureus (SA) or Candida parapsilosis (CP) by adding 0.4 mls of a 24-hour culture to 40 g of each sub-sample.

The 24-hour culture was tested to establish how much bacteria was added to the samples.

Staph. aureus: 2.1×10⁸ CFU/ml

Candida parapsilosis: 5.8×10⁴ CFU/ml

Each 100 g sub-sample inoculated with SA received roughly 2,100,000 CFU/g of sample.
Each 100 g sub-sample inoculated with CP received roughly 580 CFU/g of sample.

### Results:

TABLE 14
Total plate count results per blend for 0510001-MML:

| 0510001-MML | 0423019 SA | 0423019 CP |
|---|---|---|
| Day 0: | 1,100,000 | <10 |
| Day 1: | 190 | <10 |
| Day 2: | 50 | <10 |
| Day 3: | 160 | <10 |
| Day 4: | 30 | <10 |
| Day 5: | 50 | <10 |
| Day 6: | 40 | <10 |
| Day 7: | 80 | <10 |
| Day 14: | 50 | <10 |
| Day 21: | 160 | <10 |
| Day 28: | 80 | <10 |

Reduced to background levels by Day 2; Yeast died off at Day 0

TABLE 15
Total plate count results per blend for 0510002-MML:

| 0510002-MML | 030118 SA | 030118 CP |
|---|---|---|
| Day 0: | 2,400,000 | 430 |
| Day 1: | 1,800,000 | 360 |
| Day 2: | 400,000 | 150 |
| Day 3: | 640,000 | 170 |
| Day 4: | 500,000 | 10 |
| Day 5: | 110,000 | 30 |
| Day 6: | 120,000 | <10 |
| Day 7: | 90,000 | <10 |
| Day 14: | 660 | <10 |
| Day 21: | 300 | <10 |
| Day 28: | 400 | <10 |

Reduced to background levels by Day 21; Yeast died off by Day 6

TABLE 16
Total plate count results per blend for 0510003-MML:

| 0510003-MML | 050719 SA | 050719 CP |
|---|---|---|
| Day 0: | 3,300,000 | 200 |
| Day 1: | 560,000 | <10 |
| Day 2: | 30,000 | <10 |
| Day 3: | 47,000 | <10 |
| Day 4: | 1,000 | <10 |
| Day 5: | 2,500 | <10 |
| Day 6: | 400 | <10 |
| Day 7: | 280 | <10 |
| Day 14: | 400 | <10 |
| Day 21: | 600 | <10 |
| Day 28: | 500 | <10 |

Reduced to background levels by Day 6; Yeast died off by Day 1

TABLE 17
Total plate count results per blend for 0510004-MML:

| 0510004-MML | 043019 SA | 043019 CP |
|---|---|---|
| Day 0: | 1,500,000 | 300 |
| Day 1: | 10,000 | 190 |
| Day 2: | <10 | 40 |
| Day 3: | <10 | 10 |
| Day 4: | 10 | <10 |
| Day 5: | <10 | <10 |
| Day 6: | <10 | <10 |
| Day 7: | 30 | <10 |
| Day 14: | 130 | <10 |
| Day 21: | <10 | <10 |
| Day 28: | <10 | <10 |

Reduced to background levels by Day 2; Yeast died off by Day 4

TABLE 18
Total plate count results per blend for 0510005-MML:

| 0510005-MML | 043019 SA | 043019 CP |
|---|---|---|
| Day 0: | 510,000 | 170 |
| Day 1: | 5,000 | 200 |
| Day 2: | <10 | 120 |
| Day 3: | <10 | 70 |
| Day 4: | <10 | 20 |
| Day 5: | <10 | <10 |
| Day 6: | <10 | <10 |
| Day 7: | <10 | <10 |
| Day 14: | 30 | <10 |
| Day 21: | <10 | <10 |
| Day 28: | <10 | <10 |

Reduced to background levels by Day 2; Yeast died off by Day 5

TABLE 19
Total plate count results per blend for 0510006-MML:

| 0510006-MML | 050619 SA | 050619 CP |
|---|---|---|
| Day 0: | 700,000 | 150 |
| Day 1: | 11,000 | 170 |
| Day 2: | 10 | 30 |
| Day 3: | 10 | 20 |
| Day 4: | 10 | <10 |
| Day 5: | 20 | <10 |
| Day 6: | 20 | <10 |
| Day 7: | <10 | <10 |
| Day 14: | 150 | <10 |
| Day 21: | <10 | <10 |
| Day 28: | <10 | <10 |

Reduced to background levels by Day 2-7; Yeast died off by Day 4

TABLE 20
Total plate count results per blend for 0510007-MML:

| 0510007-MML | | |
|---|---|---|
| Botanical Emulsion Morning #4 | 050619 SA | 050619 CP |
| Day 0: | 1,100,000 | 240 |
| Day 1: | 10,000 | 110 |
| Day 2: | 30 | <10 |
| Day 3: | 10 | 30 |
| Day 4: | 10 | <10 |
| Day 5: | 30 | <10 |
| Day 6: | 20 | <10 |
| Day 7: | 10 | <10 |
| Day 14: | 20 | <10 |
| Day 21: | <10 | <10 |
| Day 28: | <10 | <10 |

Reduced to background levels by Day 2-21; Yeast died off by Day 4.

The Serum.

The serum contains 0.49% B. lactis HN019, 2.6% Essential Oils (terpenes), 1.68% Lactobacillus acidophilus NCFM (For Skin Health) and 1.09% MG 200. This data shows that by one-day significant reduction in Staph aureus and no yeast present (see FIG. 4). This formulation and process is working to reduce challenge levels.

The Exfoliant.

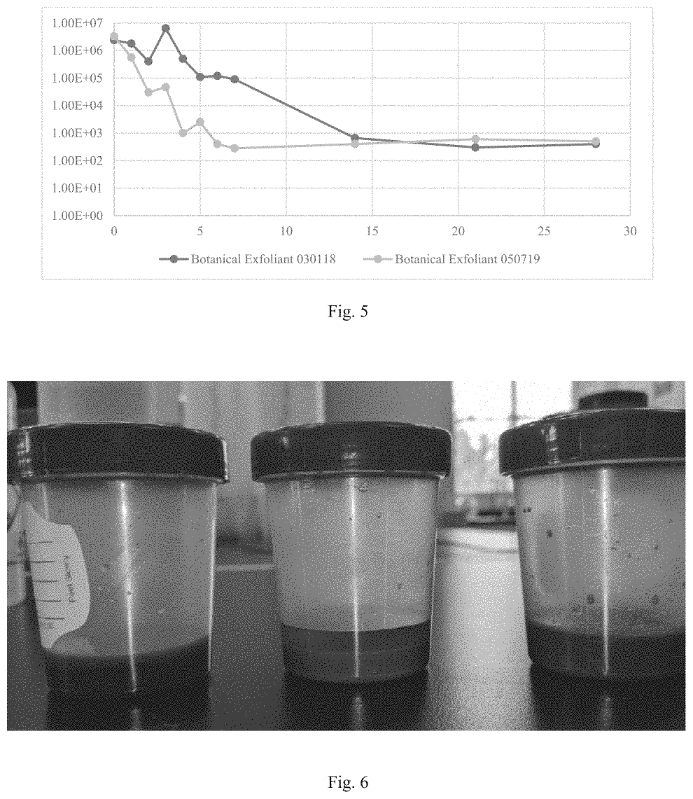

There is no water added to this formula, but due to other ingredients including clay and honey it can be considered susceptible to microbial contamination. The test results are shown in FIG. 5.

Treatment 030118—This formula contains no Bifidobacterium lactis HN019 yet was able to reduce Staph aureus background levels by day 21 and yeast absent by day 6. However, with the addition of 0.37% of Bifidobacterium lactis HN019 and MG200 at 2.1%, the background levels of Staph aureus background levels by day 6 and yeast absent by day 1. This shows that the addition of Bifidobacterium lactis HN019 and MG200 contributed significantly to the reduction of these microbial contaminants.

In addition, an unexpected quality attribute occurred with the inclusion of the probiotic and MG200 in this formula. As shown in FIG. 6, the addition of these ingredients provided a permanent and positive emulsification to the final product. Without this, the formulation quickly separates which causes an issue with dispensing this product. This is a significant quality improvement.

### The Morning and Evening Emulsions

This experiment was designed to compare morning and evening formulas with and without essential oils (formulations #1 and #3 did not contain essential oils). Additionally, there was an assessment of the efficacy of Natamycin (Morning #2 and Evening #4) on yeast results.

The emulsions all contained 0.4-0.6% Bifidobacterium lactis HN019 and 2.83% MG200. Just the Evening #1 and #2 both contained 0.0056% Natamycin. The morning emulsions did not contain Natamycin.

### Results and Discussion.

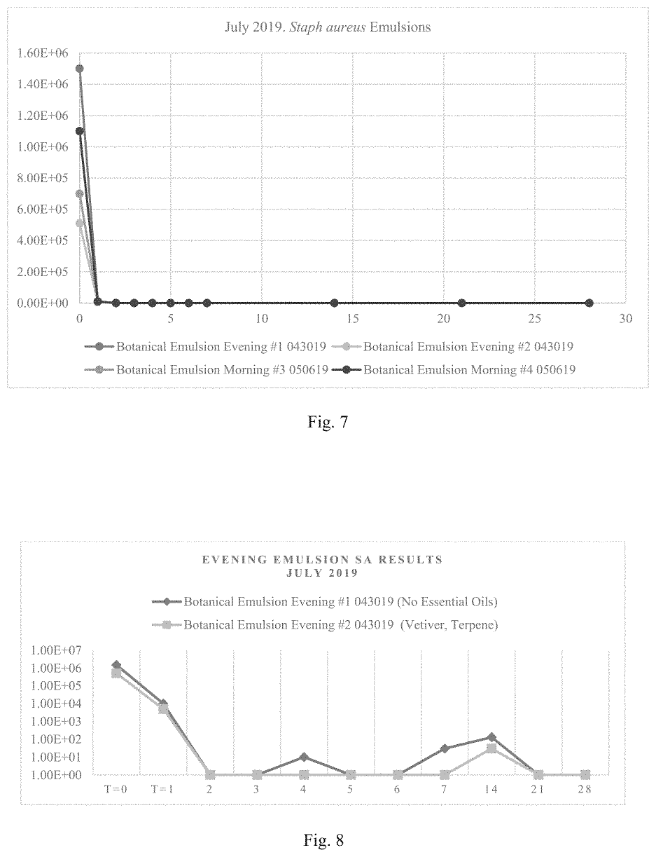

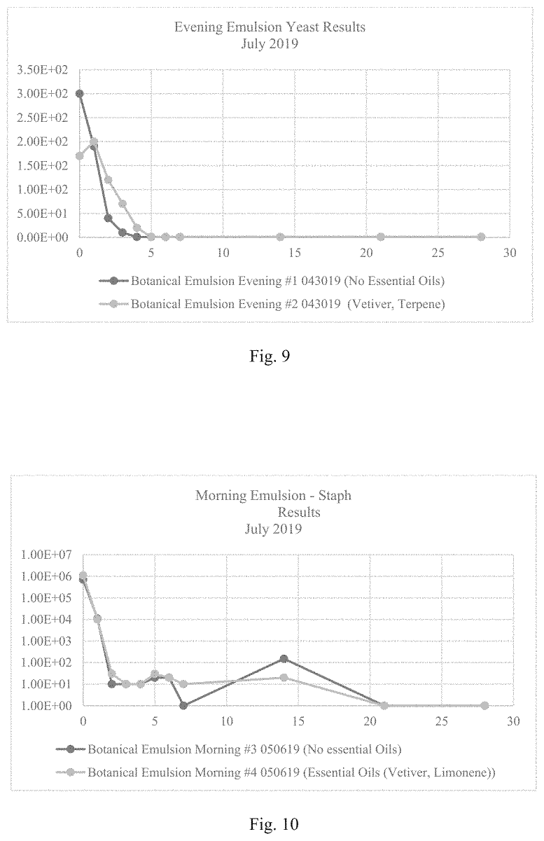

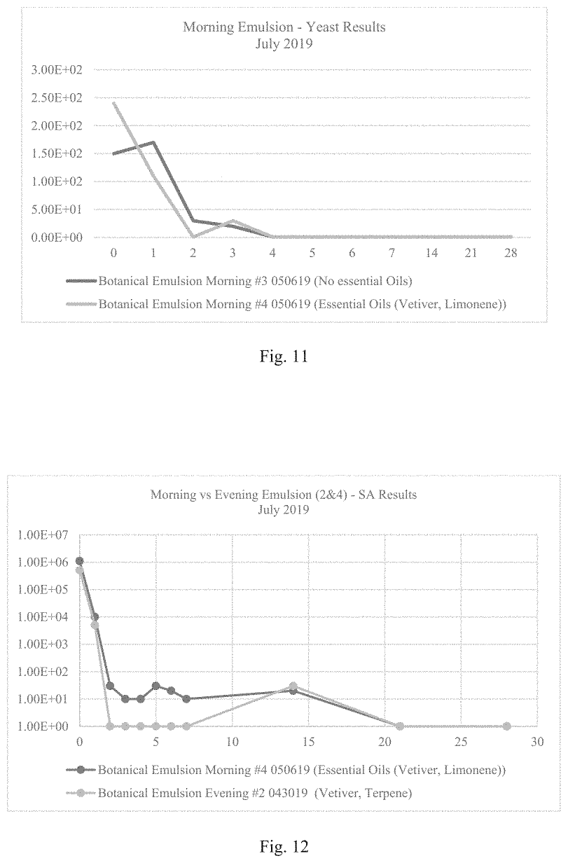

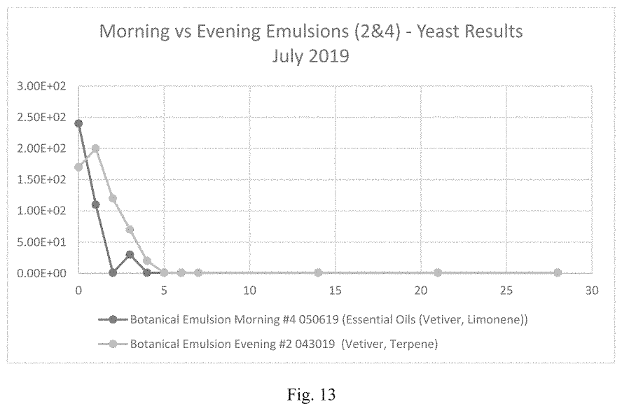

The results are shown in FIG. 7 to FIG. 13. FIG. 7 shows the Staph aureus test results of the emulsions of Example 6. FIG. 8 shows the Staph aureus test results of the evening emulsions of Example 6. FIG. 9 shows the yeast test results of the evening emulsions of Example 6. FIG. 10 shows the Staph aureus test results of the morning emulsions of Example 6. FIG. 11 shows the yeast test results of the morning emulsions of Example 6. FIG. 12 shows a comparison of the Staph aureus test results of the morning emulsion and the evening emulsion of Example 6 and the impact of natamycin. FIG. 13 shows a comparison of the yeast test results of the morning emulsion and the evening emulsion of Example 6 and the impact of essential oils.

This study found that essential oils (including terpenes) appeared to have an impact on preservation in the emulsions. They are potentially part of a hurtle-type technology developing to control microbial growth in cosmetic products. In other studies, essential oil were shown to have antimicrobial impacts. When looking at these results, botanical elements within the formulations may not be ruled out at this point for their contribution. More studies may be needed to determine the botanical elements role in contributing to this method of preservation.

Impact of emulsion temperatures. Morning #3 and #4 had emulsion temperatures of 86° F. (30° C.) and essential oils were added at 65° F. (18.3° C.). The Evening #1 and #2 had emulsion temperatures of 83° F. (28.3° C.) and essential oils were added at 65° F. (18.3° C.). The results highlight the potential impact of variations in temperature.

The Evening emulsion contains Natamycin. Natamycin is not impacting the counts, in fact the yeast counts remain higher through 14 day than the formula that does not contain Natamycin. In addition, Natamycin is contributing negatively to the odor (vinegar) and consistency of the emulsion.

We claim:

1. A method of making a composition for application to skin, comprising:
combining a raw material, water, and a first agent in a first amount effective to inhibit microbial growth to obtain a first mixture; and
mixing a second agent in a second amount effective to inhibit microbial growth and the first mixture to obtain a second mixture,
wherein a temperature of the second agent is maintained at equal to or less than about 33° C.

2. The method of claim 1, wherein the first agent comprises a fermentate.

3. The method of claim 1, wherein the first agent comprises a cultured sugar.

4. The method of claim 1, wherein the first agent comprises at least one selected from the group consisting of lactose, dextrose, fructose and maltodextrin.

5. The method of claim 1, wherein the first agent comprises an antimicrobial protein or an antimicrobial peptide.

6. The method of claim 5, wherein the first agent comprises at least one selected from the group consisting of casein, whey, soy and pea.

7. The method of claim 1, wherein the second agent comprises a probiotic.

8. The method of claim 7, wherein the probiotic comprises Bifidobacterium bacteria, Lactobacillus bacteria, Lactococcus bacteria, Streptococcus bacteria, Pediococcus bacteria, or combinations thereof.

9. The method of claim 1, wherein the first agent is a fermentate and the first amount is from about 0.75% to about 4% by weight relative to a total amount of the composition for application to skin.

10. The method of claim 1, wherein the second agent is a probiotic and the second amount is from about 0.25% to about 1% by weight relative to a total volume of the composition for application to skin.

11. The method of claim 1, further comprising mixing at least one essential oil and/or at least one terpene and the second mixture.

12. The method of claim 1, comprising:
(1) combining a fermentate and a water element to obtain a first composition;
(2) combining an oil element and a wax element, and heating to about 63° C. to about 74° C. to obtain a second composition;
(3) adding a glycerite extraction to the first composition and agitating to obtain a third composition;
(4) adding a probiotic to the third composition to obtain the fourth composition;
(5) adding the second composition that has been cooled to about 60° C., to the fourth composition and emulsifying at a temperature of less than or equal to about 33° C.;
(6) cooling the product of step (5) to less than or equal to 18° C.; and
(7) adding essential oils to the product of step (6).

13. The method of claim 1, comprising:
(1) agitating a fermentate into a liquid composition comprising water and glycerine to obtain a first composition;
(2) agitating a probiotic into the first composition to obtain the second composition; and
(3) adding at least one essential oil to the second composition.

14. The method of claim 1, comprising:
(1) combining dry ingredients, a probiotics and a fermentate to obtain a first composition;
(2) adding the first composition to a liquid component comprising water and glycerine and agitating to obtain a second composition;
(3) combining a dry plant ingredient or a clay ingredient, and the second composition, and agitating to obtain a third composition;
(4) adding a humectant to the third composition and agitating to obtain a fourth composition; and
(5) adding essential oils to the fourth composition and mixing to an incorporated consistency.

15. A composition for application to skin, comprising a first agent in a first amount effective to inhibit microbial growth and a second agent in a second amount effective to inhibit microbial growth.

16. The composition of claim 15, wherein the first agent is a fermentate and the second agent is a probiotic.

17. The composition of claim 15, wherein the second agent comprises Bifidobacterium bacteria, Lactobacillus bacteria, Lactococcus bacteria, Streptococcus bacteria, Pediococcus bacteria, or combinations thereof.

18. The composition of claim 15, wherein the second agent comprises Bifidobacterium lactis.

19. The composition of claim 15, wherein the second agent is a probiotic and a content of the probiotic in the composition for application to skin is about 0.25% to about 1% by weight as indicated by gm relative to a total volume of the composition for application to skin.

20. The composition of claim 15, wherein the first agent is a fermentate and a content of the fermentate in the composition for application to skin is about 0.75% to about 4% by weight.

21. The composition of claim 15, further comprising at least one essential oil and/or at least one terpene.

22. A method of treating acute skin infection, comprising administering an effective amount of the composition of claim 15 to a subject in need thereof.
